(19) United States
(12) Patent Application Publication
(10) Pub. No.: US 2024/0087466 A1
(43) Pub. Date: Mar. 14, 2024

(54) Method, System and Apparatus of a Video-Directed CPR Process in Cardiac Arrest

(71) Applicants: Annabelle Shen, Fort Lauderdale, FL (US); Alexander Shen, Fort Lauderdale, FL (US); Michael Y Shen, Fort Lauderdale, FL (US)

(72) Inventors: Annabelle Shen, Fort Lauderdale, FL (US); Alexander Shen, Fort Lauderdale, FL (US); Michael Y Shen, Fort Lauderdale, FL (US)

(21) Appl. No.: 18/467,535

(22) Filed: Sep. 14, 2023

Related U.S. Application Data
(60) Provisional application No. 63/406,299, filed on Sep. 14, 2022.

(51) Int. Cl. G09B 5/06; A61H 31/00; G16H 40/67; G16H 80/00

(52) U.S. Cl. CPC G09B 5/065; A61H 31/005; G16H 40/67; G16H 80/00; A61H 2201/0157; A61H 2201/502; A61H 2201/5043; A61H 2201/5048; A61H 2201/5097; A61H 2230/04; A61H 2230/207

(57) ABSTRACT

The system provides a real-time video and audio communication with a 911 call center to perform effective CPR on a patient experiencing OHCA with real-time monitoring and feedback. The system includes a Mobile App that communicates with a smart phone with camera, a wireless remote-CPR equipment stand and emergency CPR kit. The mobile app will automatically send a link to a caregiver's cell phone responsive to a 911 call placed by the caregiver, to establish a secured video call via which the 911 center can guide the caregiver in performance of CPR until an ambulance arrives.

### CROSS REFERENCE TO RELATED APPLICATIONS

This application claims the benefit of U.S. Provisional Application No. 63/406,299 filed Sep. 14, 2022 which is incorporated by reference herein in its entirety.

### TECHNICAL FIELD

This invention generally relates systems and methods for guiding lay persons to perform effective CPR during a cardiac arrest. More particularly, the invention relates to systems and methods to communicate with emergency call center and ambulance for inexperienced lay caregivers to perform CPR in an event of out of hospital cardiac arrests using a secured real-time video communication and monitoring system.

### BACKGROUND

In an event of out of hospital cardiac arrests (OHCA), effective performance of cardiopulmonary resuscitation (CPR) can determine the chance of a patient survival. The most common location of cardiac arrest occurs at home, 70% of all OHCA. Without CPR, the brain can start experiencing permanent damage within as quickly as 4 minutes and the death can occur as soon as 8 minutes. If CPR is performed immediately, the chance of survival can be doubled or tripled. However, only 46% OHCA can receive CPR and 54% of Americans do not know CPR at all. Because of lack of knowledge, delayed response and lack of adequate systems and operations, only 10% of OHCA patients are saved with 90% being fatal. Over the last several decades, calling 911 and waiting for EMT/ambulance arrival has been the routine. The average EMT response time is 7 minutes in metropolitan area, 14 minutes in rural areas, and 10% of EMT's response time is actually over 30 minutes. If the patient's family members or companions do not know CPR, such response times could prove to be fatal to many patients.

Since September 2018, the American Heart Association has recommended telephone guided CPR while waiting for ambulance arrival. However, it is very challenging for family members and companions without CPR experience to follow the audio instructions over the phone in this extremely stressful situation. How to guide family members to start an effective CPR is critical. The present inventors offer an efficient and effective system and method to guide effective CPR in real-time while waiting for ambulance arrival.

### SUMMARY

It is an object of the invention to provide a system and method to instruct a lay caregiver how to perform CPR on a patient experiencing OHCA.

It is a further object of the invention to provide a device that facilitates remote instructions of CPR techniques to a lay caregiver attending to a patient during an OHCA.

According to one aspect, the invention includes a method for providing video directed CPR instructions to a lay caregiver comprising, establishing a secure communication pathway with a caregiver's mobile device in response to a user prompt, transmitting video including a demonstration of correct CPR for display on the caregiver's mobile device via the secure communication pathway, receiving video of the caregiver performing CPR on a patient via the secure communication pathway, receiving patient chest compression information from chest compression sensors via the secure communication pathway, the chest compression information including a frequency and depth of compressions, and providing guidance to the caregiver over the secure communication pathway, the guidance being based on observations of the caregiver's performance and upon the chest compression information.

According to another aspect, the invention includes wireless enabled CPR stand comprising a mobile base; a boom extending from said mobile base, the boom including a Wi-Fi communications module and a carriage configured to attach a camera containing mobile device to the boom and a remote-control module configured to remotely control a position of the mobile device camera for improved visualization of CPR patient; and an arm extending from said boom, having first end attached to said boom and having a boom camera adjustably coupled to a second end.

### BRIEF DESCRIPTION OF THE DRAWINGS

FIG. 1 depicts a wireless CPR equipment stand in accordance with the invention.

FIG. 2A depicts a process overview of a system according to an embodiment of the invention during an OHCA.

FIG. 2B illustrates depicts a process overview of a system according to an embodiment of the invention during an OHCA.

FIG. 3 shows a CPR kit in accordance with an embodiment of the invention.

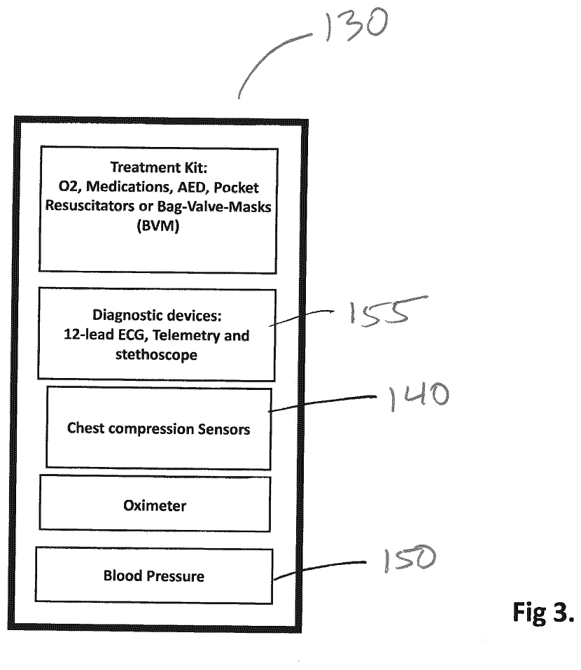

### DETAILED DESCRIPTION

The present invention encompasses a system for guiding lay persons to perform CPR on patients experiencing OHCA. The system facilitates communication between a lay caregiver, a dispatcher and/or an EMT crew. The system transmits video and patient parameters to the CPR provider or the caregiver attending to the patient. Based on the information gathered from the video information and the patient parameters, through an established audio link, i.e., a mobile phone, the CPR provider can provide audio instructions to coach the caregiver on providing appropriate CPR or other emergency care until the EMT crew arrives.

The present invention further provides a method and system with a set of apparatus for non-experienced lay persons to perform CPR in an out of hospital cardiac arrest. The system provides a real-time video and audio communication with a 911 call center to perform effective CPR with real-time monitoring and feedback. The CPR system includes a Mobile App in a smart phone with camera, wireless remote-CPR equipment stand and emergency CPR kit. The mobile app will automatically send a link to the caregiver's cell phone responsive to the 911 call, to establish a secured video call for 911 center to guide CPR while waiting for ambulance. The wireless remote CPR equipment stand has a boom camera for overall review of the patient and surroundings and a rotatable cell phone carriage to facilitate rotation of the caregiver's cell phone to allow the 911 dispatcher optimal view of the detailed CPR process and the patient. The CPR kit includes a sensor to measure and monitor depth and frequency of chest compression in real-time, an oximeter to measure and track the oxygen level as well as blood pressure cuff, a 12 lead EKG and medications for the care need of cardiac arrest.

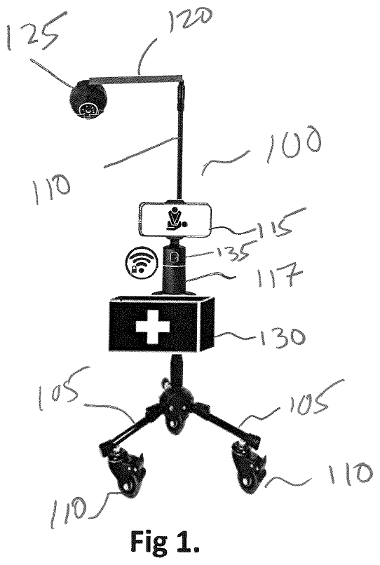

In at least one embodiment, as shown in FIG. 1, the system according to the present invention includes a Wi-Fi enabled CPR equipment stand 100 having a base including a plurality of base legs 105 where each base leg is provided with wheels 108. The stand 100 further includes a boom 110 including a mobile device carriage configured to securely hold a mobile device 115, e.g., cell phone or tablet, and a wireless remote-control module 117. CPR equipment stand 100 is further provided with a camera arm 120 having a boom camera 125, e.g., a 3D camera with a 360° field of view, attached thereto. The position of the 3D camera 125 may be controlled by the CPR provider via the wireless remote-control module 117 to provide optimum visualization of the CPR process. An CPR kit 130 containing medications and equipment necessary for treating the patient prior to ambulance arrival is also provided. The CPR equipment stand 100 may be Wi-Fi installed and enabled to ensure smooth connection between the dispatcher, the EMT crew and the layperson for visualization of the process without setbacks.

In some embodiments, an emergency CPR kit 130 is provided which is configured for attachment to boom 110. Emergency CPR Kit 130 contains sensors, testing devices, and medications which a caregiver may apply to an OHCA patient to improve survivability while waiting for EMT personnel to arrive. For example, Emergency CPR kit 130 may include chest compression sensors 140 such as position accelerators used to track the frequency and depth of chest compressions. Suitable sensors include position sensors, such as linear, rotary and angular position sensors. Chest compression sensors 140 are configured to continuously transmit data wirelessly thereby allowing the dispatcher and/or the EMT crew to be apprised of the patient's status in real time. Emergency CPR kit 130 may further include pulse oximeters 145 configurable to be clipped onto the ear of the patient so as to check oxygen concentration in blood and verify that adequate blood flow reaches the brain.

In some embodiments, CPR kit 130 may be attached to boom 110 via a kit carriage (not shown). In other embodiments, CPR kit 130 may be detached from boom 110.

To facilitate communication and data transmission, with the dispatcher and the EMT crew, stand 100 may be Wi-Fi installed and enabled. To that end, boom 110 is provided with Wi-Fi communication module 135 which permits wireless communication between devices.

Emergency CPR kit 130 may further include testing devices such as a blood pressure monitor 150, a wireless 12-lead ECG 155 and telemetry allowing the lay caregiver to monitor and diagnose the presence of an acute heart attack and arrhythmia on site with the aid of instruction from the dispatcher and/or EMT crew. Emergency CPR kit 130 further includes oxygen and medications, e.g., epinephrine, atropine, lidocaine etc., A medication kit such as that described in U.S. Pat. No. 10,980,417 B2 may also be employed. Such medications provide for immediate treatment of acute heart attack and arrhythmia and can be administered before ambulance arrival. This would improve survival especially in remote areas or extremely crowded cities where the average EMT response time is long. The medications may be refrigerated at low temperatures and systemically organized for easy reference whether using colors, numbers, or special marks to differentiate types of medications. In addition, computing devices may be provided to track/store expiration dates of the medications stored in CPR kit 130 to avoid administering stale medications.

In some embodiments, emergency CPR kit 130 includes other devices such as an automated external defibrillator (AED), an external pacemaker, and ventilation masks. Additionally, basic first aid devices such as needles and IV tubing and personal protective equipment such as gloves, clean sheets, and towels are provided.

In keeping with the invention, a mobile app is provided which may be installed on the caregiver's mobile device or which may run on a remote computing device. An appropriate mobile device must include a camera and have the ability to make and receive calls. In response to a call from the caregiver, in some embodiments, the dispatcher can send an encrypted (HIPPAA compliant for patient privacy) video call link to the caregiver's mobile device. When the link is activated by the caregiver, a secure communication path is created which allows transmission of data to and from the dispatcher and the caregiver's mobile device. Accordingly, the dispatcher can present instructions to the caregiver over the secure communication path. For example, the dispatcher may perform a demonstration on a mannequin showing proper CPR techniques, i.e., using correct hands position at the correct chest location for the caregiver to perform, relative to the patient through the caregiver's mobile device screen in real time. A mobile app may be provided to be installed on the caregiver's mobile device. The mobile app can connect with chest compression sensors 140, pulse oximeters 145, blood pressure monitors and the 12-lead ECG and transmit data from those devices to the dispatcher and the EMT crew. Accordingly, the dispatcher can assess the chest compression's effectiveness based on the frequency and depth from compression sensors 140 and provide standard CPR instruction and individualized guidance based on the real time feedback.

Based on the caregiver's performance, the dispatcher can suggest that compressions proceed faster or harder for better effectiveness to meet AHA guidelines of 100-120 compressions per minute and a depth of 1½ to 2 inches. In some embodiments, in addition to or in lieu of the verbal instructions provided by the dispatcher, the mobile app may generate audible prompts to pace CPR chest compression, the prompts being representative of a compression rate of between 100 to 120 compressions per minute. The mobile app of the video may further automatically track facial expression and body motion of the patient during CPR. Recent developments of remote non-touching assessments of pulse rate, oxygen level and blood pressure can be provided via videos in real-time to reflect the effectiveness of the chest compression in addition to the measurement of the caregiver efforts. The combination of real-time measurements of mechanical efforts and the actual physiological results can provide critical information for the 911 dispatcher and the EMT crew to help guide the caregiver for more effective CPR compression. Additional tools with drones can also be sent to the site for further assistance, such as AED, emergency meds, etc.

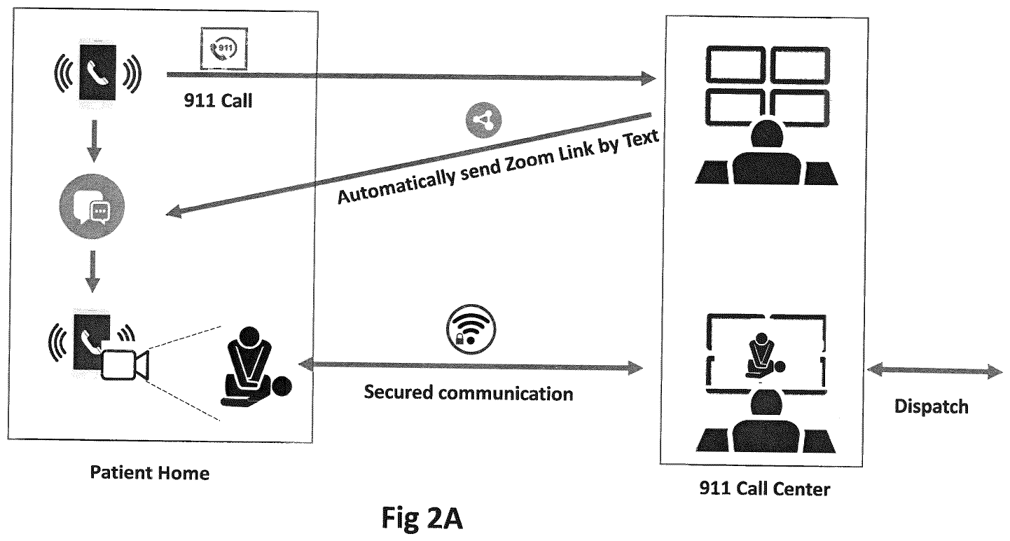

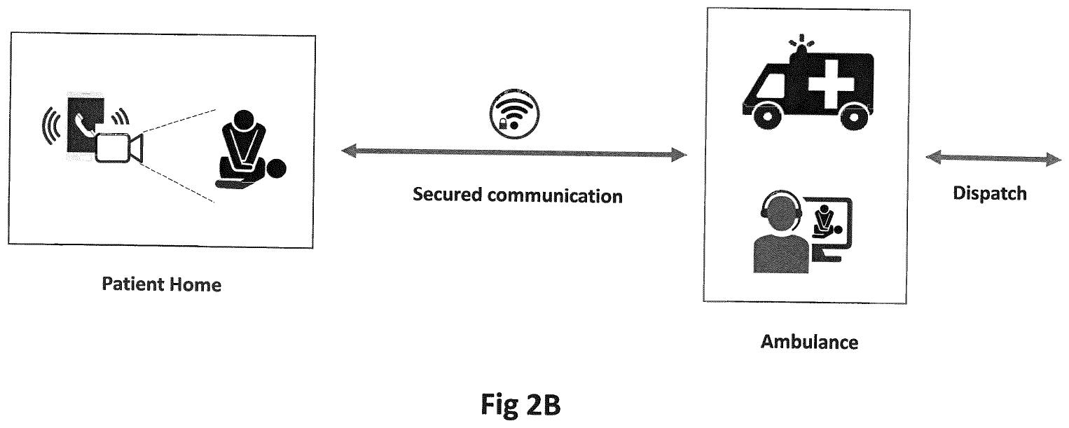

FIGS. 2A and 2B illustrate operational aspects of the systems discussed herein. When an OHCA occurs, a Caregiver can call 911 using a mobile device provided with a camera such as a smartphone. The 911 dispatcher may then send a secured video call link back to the caregiver's mobile device and establishes a connection to the CPR equipment stand and activates all related devices and sensors discussed above. The caregiver can then click the link to establish the video call and mount the mobile device to the CPR equipment stand. As discussed above, the dispatcher can remotely rotate and control the mobile device camera for the optimal viewing of the patient and the CPR process. The boom camera mounted on the CPR equipment stand can also provide wide range of view, i.e., up to 360°.

On the video call, the dispatcher perform a demonstration on a mannequin showing how to (1) use the chest compression sensor, (2) mount the oximeter on the ear, (3) properly position the caregiver relative to the patient, (4) cross hands, (5) properly lock shoulders to perform CPR. If additional caregiver(s) are available, the dispatcher can instruct one caregiver to start CPR immediately and guide the other caregiver(s) to put the sensors on, since the timing of the chest compression is critically important to the patient's survival.

Based on the feedback from the video/audio communication as well as the various sensors, the dispatcher can keep track of patient's vitals and make sure it is within range of physician's advice. The same or a different dispatcher can dispatch an ambulance, and provide the location where the patient is experiencing the OHCA as well as the video link to allow the EMT crew to participate in the CPR instruction.

In accordance with a feature of the invention, a computer management system may be provided with tracking during an event and reporting post event along with a database to record all CPR kit use in each CPR event as well as outcomes. The actual performance can be compared to current CPR operation standards to identify the gaps for further improvements. Each CPR kit software, App and instructions may be updated in version controls to keep all measurement device performance/maintenance and instruction guidelines current on, e.g., vitals, monitoring and treatment accessories, as well as AI analysis of vital sensing, facial/body movement, intervention updates, as well as standard operation updates. Further, An AI system can be connected to the computer management system for machine learning or other mechanisms to improve the system function and performance over time.

In at least one embodiment the sensors described herein may be attached sensors or remote sensors as shown in the below table.

| ATTACHED SENSORS | REMOTE SENSORS |
|---|---|
| Inertial measurement units | Lidar sensors - optimal distance |
| (IMUs) - capture and measure | can range from a few centimeters to |
| accelerations and angular rates | hundreds of meters |
| Gyroscopes - measure angular rate | Ultrasonic sensors - optimal |
| or orientation | distance up to a few meters |
| Accelerometers - measure | Time-of-flight sensors - optimal |
| acceleration | distance ranges from a few |
| | centimeters to a few meters. |
| Capacitive sensors - can be | Infrared proximity sensors - optimal |
| embedded in wearable devices to | distance usually in the range of a |
| detect proximity or touch | few centimeters |
| Stretch and bend sensors - change | Laser distance sensors - optimal |
| resistance as they are stretched or | distance from between a few |
| bent | millimeters to dozens of meters |

While the invention has been described with reference to certain embodiments, numerous changes, alterations and modifications to the described embodiments are possible without departing from the spirit and scope of the invention as defined in the appended claims and equivalents thereof.

Although the present invention has been described in terms of particular embodiments, it is not limited to those embodiments. Alternative embodiments, examples, and modifications which would still be encompassed by the invention may be made by those skilled in the art, particularly in light of the foregoing teachings. The example and alternative embodiments described above may be combined in a variety of ways with each other without departing from the invention.

Those skilled in the art will appreciate that various adaptations and modifications of the embodiments described above can be configured without departing from the scope and spirit of the invention. Therefore, it is to be understood that, within the scope of the appended claims, the invention may be practiced other than as specifically described herein.

1. A method for providing video directed CPR instructions to a lay caregiver comprising:
establishing a secure communication pathway with a caregiver's mobile device in response to a user prompt;
transmitting video including a demonstration of correct CPR for display on the caregiver's mobile device via said secure communication pathway;
receiving video of the caregiver performing CPR on a patient via said secure communication pathway;
receiving patient chest compression information from chest compression sensors via the secure communication pathway, the chest compression information including a frequency and depth of compressions;
providing guidance to the caregiver over the secure communication pathway, the guidance being based on observations of the caregiver's performance and upon the chest compression information.

2. The method of claim 1 further comprising receiving patient parameters including at least one of pulse reading, pulse oximeter reading, blood pressure reading of the patient and ECG reading of the patient and wherein providing audio guidance includes providing guidance based upon the at least one received patient parameter.

3. The method of claim 1 wherein receiving video of the caregiver performing CPR on the patient includes video of the caregiver's hand position and the patient's face and video of the caregiver's position relative to the patient's body.

4. The method of claim 1 including transmitting an audible prompt to pace CPR chest compression, said prompt being representative of an instructed compression rate.

5. The method of claim 1 wherein the instructed compression rate is between 100 to 120 compressions per minute.

6. The method of claim 1 further comprising establishing a communication pathway for video communication between caregivers at an event site, 911 dispatch center, ambulance and hospital for better support and preparation for further resuscitation, on-site emergency care and transfer to the hospital.

7. A wireless enabled CPR stand comprising:
a mobile base;
a boom extending from said mobile base, said boom including a Wi-Fi communications module and a carriage configured to attach a camera containing mobile device to said boom and a remote-control module configured to remotely control a position of the mobile device camera for improved visualization of CPR patient; and
an arm extending from said boom, having first end attached to said boom and having a boom camera adjustably coupled to a second end.

8. The wireless enabled CPR stand of claim 7 wherein a position of the camera is wirelessly adjustable.

9. The wireless enabled CPR stand of claim 6 wherein the boom camera provides full visualization of a cardiac arrest site entire environment thereby allowing for better instructions and communications with caregivers;

10. The wireless enabled CPR stand of claim 7 further comprising a CPR kit containing a wireless chest compression sensor.

11. The wireless enabled CPR stand of claim 10 wherein said CPR kit contains pulse oximeters.

12. The wireless enabled CPR stand of claim of claim 10 wherein said CPR kit contains a wireless 12-lead ECG.

13. The wireless enabled CPR stand of claim 10 wherein said CPR kit contains a blood pressure measuring device.

14. The wireless enabled CPR stand of claim 10 wherein said boom includes a carriage configure to attach said CPR kit to said boom.

15. The wireless enabled CPR stand of claim 7 wherein said mobile base includes a plurality of legs having attached wheels.

16. The System of claim 13 wherein the CPR kit includes at least one of a wireless pulse rate sensor, a pulse oximeter, a blood pressure measurement device.
